(19) United States
(12) Patent Application Publication
(10) Pub. No.: US 2022/0151883 A1
(43) Pub. Date: May 19, 2022

(54) OILY SOLID COSMETIC, METHOD FOR PRODUCING THE OILY SOLID COSMETIC, AND METHOD FOR PRODUCING MOLDED PRODUCT USING THE OILY SOLID COSMETIC

(71) Applicant: Miyauchi Co., Ltd., Kanagawa (JP)

(72) Inventors: Noriaki KADOHAMA, Kanagawa (JP); Kenichi TAIRA, Kanagawa (JP)

(73) Assignee: Miyauchi Co., Ltd., Kanagawa (JP)

(21) Appl. No.: 17/527,377

(22) Filed: Nov. 16, 2021

(30) Foreign Application Priority Data
Nov. 17, 2020 (JP) 2020-191152

(51) Int. Cl. A61K 8/02; A61K 8/37; A61K 8/25; A61K 8/29; A61K 8/19; A61K 8/92; A61Q 19/00; A61K 8/34; A61K 8/31; A61K 8/891

(52) U.S. Cl. CPC A61K 8/0216; A61K 8/375; A61K 8/25; A61K 8/29; A61K 8/19; A61K 2800/43; A61K 8/37; A61Q 19/00; A61K 8/34; A61K 8/31; A61K 8/891; A61K 8/922

(57) ABSTRACT

The present invention provides an oily solid cosmetic having excellent fixability to the skin, high clarity of application color, and including a powder of a base material that is conspicuous on the skin without whitening.

The oily solid cosmetic of the present invention is configured to include a cosmetic base material obtained by mixing a powder (A) and an oily agent (B), and a low viscous liquid oil (C) that is not volatile at normal temperature for rendering muddy the cosmetic base material.

### TECHNICAL FIELD

The present invention relates to an oily solid cosmetic having excellent fixability to the skin, high clarity of application color, and including a powder of a base material that is conspicuous on the skin without whitening, a method for producing the oily solid cosmetic, and a method for producing a molded product using the oily solid cosmetic.

### BACKGROUND

Conventionally, powder solid cosmetics obtained by pressing and filling a muddy cosmetic are widely used as eye shadow, eyebrow, cheek makeup, foundation, lipstick cosmetics and so forth.

Powder solid cosmetics thus obtained provide favorable tight adhesion to the skin and excellent color development as well, but are poorer in these properties than creamy oily solid cosmetics. In addition, since such cosmetics can impart a color tone from a white powder of talc or mica to be blended as a base material ingredient, it is extremely hard to achieve transparent finish as part of an application color.

Meanwhile, it is difficult to obtain cosmetics having a good feel because creamy oily solid cosmetics provide favorable tight adhesion to the skin, but the stickiness is high due to a high content of oily ingredients.

Also, conventional methods for rendering cosmetics muddy with a volatile solvent such as water and alcohol require a step of drying the volatile solvent after pressing and filling cosmetics.

Patent Document 1 proposes an elastic silicone-based oily solid cosmetic including a powder ingredient in an amount of 40 to 70% by mass, the powder ingredient containing a sparkling powder, a silicone wax with a melting point of 25 to 55° C., and a crosslinked silicone elastomer.

Patent Document 2 proposes a gel-like composition, including an oily agent and a silicone elastic powder dispersed substantially in a state of a primary particle in the oily agent, in which the gel-like composition has a storage modulus at 25° C. of 50 to 500 Pa with a ratio of loss modulus/storage modulus of 1.0 or less and has a storage modulus at 80° C. of 1 to 50 Pa with a ratio of loss modulus/storage modulus of 1.0 or more when the stress is 1 Pa.

Patent Document 3 proposes solid powder cosmetics containing a powder phase component, an oil phase component, hydrophobic polysaccharide and water, the hydrophobic polysaccharide component being contained in an amount of 0.1 to 0.15 parts by mass with respect to the total of 100 parts by mass of the powder phase component and the oil phase component, and the water component being the residual amount of the water added as a dispersion medium during production.

Patent Document 4 proposes a solid powder cosmetic obtained by mixing a cosmetic base material containing a powder surface-coated with a surface coating treatment agent such as a both terminal reactive diorganopolysiloxane and an amino group-containing silane compound and a liquid oily ingredient at 25° C. with one or more combined solvents selected from water and aqueous solvents to prepare a slurry mixture, filling the mixture into a container, and removing the solvent.

However, all the Patent Documents 1 to 4 fail to disclose or suggest technologies of obtaining an oily solid cosmetic having excellent fixability to the skin, high clarity of application color, and including a powder of a base material that is conspicuous on the skin without whitening using a powder (A), an oily agent (B) and a low viscous liquid oil (C), without using creamy solidified ingredients or volatile ingredients such as volatile solvents.

### CITATION LIST

### Patent Literature

Patent Literature 1: JP 2020-138921 A
Patent Literature 2: JP 2006-057054 A
Patent Literature 3: JP 2020-164428 A
Patent Literature 4: JP 2018-168130 A

### SUMMARY OF INVENTION

### Technical Problem

Thus, the present invention was made in view of the situation to solve the problems, and has an object to provide an oily solid cosmetic having excellent fixability to the skin, high clarity of application color, and including a powder of a base material that is conspicuous on the skin without whitening, a method for producing the oily solid cosmetic, and a method for producing a molded product using the oily solid cosmetic.

### Solution to Problem

An oily solid cosmetic according to the present invention is most essentially configured to include a cosmetic base material obtained by mixing a powder and an oily agent, and a low viscous liquid oil that is not volatile at normal temperature for rendering muddy the cosmetic base material.

### Advantageous Effects of Invention

The invention according to claim 1 can achieve and provide an oily solid cosmetic having excellent fixability to the skin, high clarity of application color, and including a powder of a base material that is conspicuous on the skin without whitening.

The invention according to claim 2 can achieve and provide a colored oily solid cosmetic having excellent fixability to the skin, high clarity of application color, and including a powder of a base material that is conspicuous on the skin without whitening.

The invention according to claim 3 can achieve and provide the oily solid cosmetic having the advantageous effect of the invention according to claim 2, in which a powder including a coloring agent is blended in an amount of 20 to 65% by weight, an oily agent in an amount of 5 to 20% by weight, and a low viscous liquid oil that is not volatile at normal temperature in an amount of 30 to 60% by weight.

The invention according to claim 4 can achieve and provide a production method capable of obtaining the oily solid cosmetic having the advantageous effect of the invention according to claim 1, including the steps of: mixing a powder and an oily agent to obtain a cosmetic base material; and adding to the cosmetic base material a low viscous liquid oil that is not volatile at normal temperature, and stirring the resulting mixture to render muddy the cosmetic base material.

The invention according to claim 5 can achieve and provide a production method capable of obtaining the oily solid cosmetic having the advantageous effect of the invention according to claim 2, including the steps of: mixing a powder and an oily agent to obtain a cosmetic base material, the powder including a coloring agent; and adding to the cosmetic base material a low viscous liquid oil that is not volatile at normal temperature, and stirring the resulting mixture to render muddy the cosmetic base material.

The invention according to claim 6 can achieve and provide a production method having the similar advantageous effect of the invention according to claim 5, in which the powder including a coloring agent is blended in an amount of 20 to 65% by weight, the oily agent in an amount of 5 to 20% by weight, and the low viscous liquid oil that is not volatile at normal temperature in an amount of 30 to 60% by weight.

The invention according to claim 7 can achieve and provide a production method capable of obtaining a molded product suitable as eye shadow, eyebrow, cheek makeup, foundation, lipstick cosmetics and so forth, using the oily solid cosmetic having the above-mentioned advantageous effect of the invention.

The invention according to claim 8 can achieve and provide a production method capable of obtaining a molded product, using a colored oily solid cosmetic suitable as eye shadow, eyebrow, cheek makeup, foundation, lipstick cosmetics and so forth, in which a powder including a coloring agent is blended in an amount of 20 to 65% by weight, an oily agent in an amount of 5 to 20% by weight, and a low viscous liquid oil that is not volatile at normal temperature in an amount of 30 to 60% by weight, and a cosmetic base material obtained by mixing the powder including a coloring agent and the oily agent is rendered muddy with the low viscous liquid oil that is not volatile at normal temperature, and filled into a container as the low viscous liquid oil is removed.

The invention according to claim 9 can achieve and provide a production method capable of obtaining a molded product using the oily solid cosmetic having the advantageous effect of the invention according to claim 8 in a back injection system (filling from the rear surface of a container) with a viscosity of the low viscous liquid oil of 200 cSt or less when the oily solid cosmetic is filled from holes having a small diameter provided on the bottom of the container or the like.

The invention according to claim 10 can achieve and provide a production method capable of obtaining a molded product using the oily solid cosmetic having the advantageous effect of the invention according to claim 8 in a front injection system (filling from the front surface of a container) with a viscosity of the low viscous liquid oil of 500 cSt or less when the oily solid cosmetic is filled from an upper portion of the container and pressed.

### BRIEF DESCRIPTION OF DRAWINGS

FIG. 1 is an explanatory diagram showing blending ratios for elements, evaluation items, and evaluation results regarding Examples 1 to 6, and Comparative Examples 1 and 2 according to the present invention.

### DESCRIPTION OF EMBODIMENTS

The present invention has achieved an object of providing an oily solid cosmetic having excellent fixability to the skin, high clarity of application color, and including a powder of a base material that is conspicuous on the skin without whitening, the oily solid cosmetic including a cosmetic base material obtained by mixing a powder and an oily agent, the powder including a coloring agent, and a low viscous liquid oil that is not volatile at normal temperature for rendering muddy the cosmetic base material, in which the powder including a coloring agent is blended in an amount of 20 to 65% by weight, the oily agent in an amount of 5 to 20% by weight, and the low viscous liquid oil that is not volatile at normal temperature in an amount of 30 to 60% by weight.

### EXAMPLES

An oily solid cosmetic according to Examples of the present invention, a method for producing the oily solid cosmetic, and a method for producing a molded product using the oily solid cosmetic will be described in detail with reference to the drawing.

The oily solid cosmetic of the Examples includes a cosmetic base material obtained by mixing a powder (A) and an oily agent (B), the powder including a coloring agent, and a low viscous liquid oil (C) that is not volatile at normal temperature for rendering muddy the cosmetic base material.

The powder (A) used is one or a mixture of two or more of commonly-used cosmetic ingredients such as talc, mica, synthetic mica, sericite, titanium oxide, boron nitride, silica, silicone powder, nylon powder, and PMMA, and the coloring agent ingredient used is iron oxide, azurite, Prussian blue, an organic color pigment such as Red No. 202, black iron oxide-coated mica titanium, red iron oxide-coated mica titanium, carbon black, glass powder, aluminum powder, and so forth.

The powder (A) including the coloring agent ingredient is blended in an amount of 20 to 65% by weight.

The oily agent (B) used is, for example, one or a mixture of two or more of isostearic acid hydrogenated castor oil, isostearic acid sorbitan, Vaseline, cholesteryl hydroxystearate, diisostearyl malate, phytosteryl macadamiate, polyethylene, ceresin, microcrystalline wax, synthetic wax, bees wax, carnauba wax, candelilla wax, and so forth.
The oily agent (B) is blended in an amount of 5 to 20% by weight.

The low viscous liquid oil (C) used is, for example, one or a mixture of two or more of liquid paraffin, dimeticone, squalane, isotridecyl isononanate, neopentylglycol dicaprylate, and so forth.

The low viscous liquid oil (C) is blended in an amount of 30 to 60% by weight.

In addition to the above-mentioned ingredients, commonly-used cosmetic ingredients can obviously be blended.

For example, a perfume, a dispersant, a UV absorber, a stabilizing agent, an antioxidant, a pH modifier, a thickener, a preservative, a cosmetic component or the like can be blended.

The low viscous liquid oil (C) used may be a volatile oily agent.

Specific Examples 1 to 6, and Comparative Examples 1 and 2 of the oily solid cosmetic according to the Examples will be described.

### Example 1

A powder (A) and a coloring agent ingredient are blended in a total amount of 20 to 65% by weight, an oily agent (B) is in an amount of 5 to 20% by weight, a low viscous liquid oil (C) is in an amount of 30 to 65% by weight, and using a mixer or a Henschel mixer commonly used in the production of cosmetic products, the powder (A) including the coloring agent ingredient and the oily agent (B) are mixed, the low viscous liquid oil (C) is added to the resulting mixture (blended in an amount of 35%), and stirred by a mixer, a disperser, a homomixer or the like to obtain a muddy cosmetic.

Specifically, the powder (A) is one or a mixture of two or more of commonly-used cosmetic product materials such as talc (9%), mica (10%), synthetic mica (15%) or the like, the coloring agent ingredient is iron oxide (5%), black iron oxide-coated mica titanium (5%), or red iron oxide-coated mica titanium (5%), the oily agent (B) is isostearic acid hydrogenated castor oil (3%), stearic acid sorbitan (3%), or diisostearyl malate (10%), and the low viscous liquid oil (C) is triethylhexanoin (35%).

Additionally, the powder (A) can be blended such as sericite, titanium oxide, boron nitride, a silicone powder, a nylon powder, PMMA or the like, and the coloring agent ingredient can be blended such as azurite, Prussian blue, an organic color pigment like Red No. 202, carbon black, a glass powder, an aluminum powder, and so forth.

The oily agent (B) can be, for example, one or a mixture of two or more of Vaseline, cholesteryl hydroxystearate, phytosteryl macadamiate, polyethylene, ceresin, microcrystalline wax, synthetic wax, bees wax, carnauba wax, candelilla wax, and so forth.

### Comparative Example 1

A powder (A) and a coloring agent ingredient are blended in a total amount of 20 to 65% by weight, an oily agent (B) is in an amount of 5 to 20% by weight, a low viscous liquid oil (C) is in an amount of 30 to 65% by weight, and using a mixer or a Henschel mixer commonly used in the production of cosmetic products, the powder (A) including the coloring agent ingredient and the oily agent (B) are mixed.

The low viscous liquid oil (C) is added to the resulting mixture (blended in an amount of 35%) and stirred by a mixer, a disperser, a homomixer or the like to obtain a muddy cosmetic.

Specifically, the powder (A) is one or a mixture of two or more of commonly used cosmetic product materials such as talc (9%), mica (10%), synthetic mica (15%) or the like, the coloring agent ingredient is iron oxide (5%), black iron oxide-coated mica titanium (5%), or red iron oxide-coated mica titanium (5%), the oily agent (B) is isostearic acid hydrogenated castor oil (3%), stearic acid sorbitan (3%), or diisostearyl malate (10%), and the low viscous liquid oil (C) is ethanol (35%).

### Comparative Example 2

Comparative Example 2 is substantially similar to Comparative Example 1.

However, the low viscous liquid oil (C) used is isopropanol (35%) in place of ethanol (35%) to obtain a muddy cosmetic as in Comparative Example 1.

### Example 2

A powder (A) and a coloring agent ingredient are blended in a total amount of 20 to 65% by weight, an oily agent (B) is in an amount of 5 to 20% by weight, a low viscous liquid oil (C) is in an amount of 30 to 65% by weight, and using a mixer or a Henschel mixer commonly used in the production of cosmetic products, the powder (A) including the coloring agent ingredient and the oily agent (B) are mixed, the low viscous liquid oil (C) is added to the resulting mixture (blended in an amount of 35%), and stirred by a mixer, a disperser, a homomixer or the like to obtain a muddy cosmetic.

Specifically, the powder (A) is one or a mixture of two or more of commonly used cosmetic product materials such as talc (9%), mica (10%), synthetic mica (15%) or the like, the coloring agent ingredient is iron oxide (5%), black iron oxide-coated mica titanium (5%), or red iron oxide-coated mica titanium (5%), the oily agent (B) is isostearic acid hydrogenated castor oil (3%), stearic acid sorbitan (3%), or diisostearyl malate (10%), and the low viscous liquid oil (C) is liquid paraffin (35%).

Comparative Examples 1 and 2 are the same as the above-described Examples.

### Example 3

A powder (A) and a coloring agent ingredient are blended in a total amount of 20 to 65% by weight, an oily agent (B) is in an amount of 5 to 20% by weight, a low viscous liquid oil (C) is in an amount of 30 to 65% by weight, and using a mixer or a Henschel mixer commonly used in the production of cosmetic products, the powder (A) including the coloring agent ingredient and the oily agent (B) are mixed, the low viscous liquid oil (C) is added to the resulting mixture (blended in an amount of 35%), and stirred by a mixer, a disperser, a homomixer or the like to obtain a muddy cosmetic.

Specifically, the powder (A) is one or a mixture of two or more of commonly used cosmetic product materials such as talc (9%), mica (10%), synthetic mica (15%) or the like, the coloring agent ingredient is iron oxide (5%), black iron oxide-coated mica titanium (5%), or red iron oxide-coated mica titanium (5%), the oily agent (B) is isostearic acid hydrogenated castor oil (3%), stearic acid sorbitan (3%), or diisostearyl malate (10%), and the low viscous liquid oil (C) is dimeticone (35%).

Comparative Examples 1 and 2 are the same as the above-described Examples.

### Example 4

A powder (A) and a coloring agent ingredient are blended in a total amount of 20 to 65% by weight, an oily agent (B) is in an amount of 5 to 20% by weight, a low viscous liquid oil (C) is in an amount of 30 to 65% by weight, and using a mixer or a Henschel mixer commonly used in the production of cosmetic products, the powder (A) including the coloring agent ingredient and the oily agent (B) are mixed, the low viscous liquid oil (C) is added to the resulting mixture (blended in an amount of 35%), and stirred by a mixer, a disperser, a homomixer or the like to obtain a muddy cosmetic.

Specifically, the powder (A) is one or a mixture of two or more of commonly used cosmetic product materials such as talc (9%), mica (10%), synthetic mica (15%) or the like, the coloring agent ingredient is iron oxide (5%), black iron oxide-coated mica titanium (5%), or red iron oxide-coated mica titanium (5%), the oily agent (B) is isostearic acid hydrogenated castor oil (3%), stearic acid sorbitan (3%), or diisostearyl malate (10%), and the low viscous liquid oil (C) is squalane (35%).

Comparative Examples 1 and 2 are the same as the above-described Examples.

### Example 5

A powder (A) and a coloring agent ingredient are blended in a total amount of 20 to 65% by weight, an oily agent (B) is in an amount of 5 to 20% by weight, a low viscous liquid oil (C) is in an amount of 30 to 65% by weight, and using a mixer or a Henschel mixer commonly used in the production of cosmetic products, the powder (A) including the coloring agent ingredient and the oily agent (B) are mixed, the low viscous liquid oil (C) is added to the resulting mixture (blended in an amount of 35%), and stirred by a mixer, a disperser, a homomixer or the like to obtain a muddy cosmetic.

Specifically, the powder (A) is one or a mixture of two or more of commonly used cosmetic product materials such as talc (9%), mica (10%), synthetic mica (15%) or the like, the coloring agent ingredient is iron oxide (5%), black iron oxide-coated mica titanium (5%), or red iron oxide-coated mica titanium (5%), the oily agent (B) is isostearic acid hydrogenated castor oil (3%), stearic acid sorbitan (3%), or diisostearyl malate (10%), and the low viscous liquid oil (C) is isotridecyl isononanoate (35%).

Comparative Examples 1 and 2 are the same as the above-described Examples.

### Example 6

A powder (A) and a coloring agent ingredient are blended in a total amount of 20 to 65% by weight, an oily agent (B) is in an amount of 5 to 20% by weight, a low viscous liquid oil (C) is in an amount of 30 to 65% by weight, and using a mixer or a Henschel mixer commonly used in the production of cosmetic products, the powder (A) including the coloring agent ingredient and the oily agent (B) are mixed, the low viscous liquid oil (C) is added to the resulting mixture (blended in an amount of 35%), and stirred by a mixer, a disperser, a homomixer or the like to obtain a muddy cosmetic.

Specifically, the powder (A) is one or a mixture of two or more of commonly used cosmetic product materials such as talc (9%), mica (10%), synthetic mica (15%) or the like, the coloring agent ingredient is iron oxide (5%), black iron oxide-coated mica titanium (5%), or red iron oxide-coated mica titanium (5%), the oily agent (B) is isostearic acid hydrogenated castor oil (3%), stearic acid sorbitan (3%), or diisostearyl malate (10%), and the low viscous liquid oil (C) is neopentyl glycol dicaprate (35%).

Comparative Examples 1 and 2 are the same as the above-described Examples.

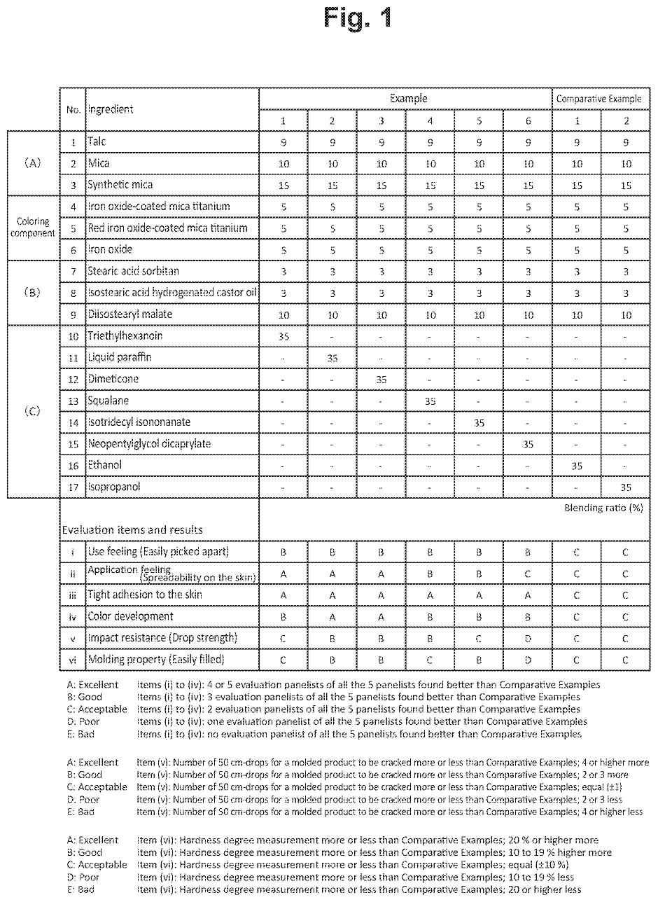

FIG. 1 shows blending ratios for elements, evaluation items, and evaluation results regarding Examples 1 to 6, and Comparative Examples 1 and 2.

As clearly shown in FIG. 1, there are 6 evaluation items: (i) use feeling (easily picked apart), (ii) application feeling (spreadability on the skin), (iii) tight adhesion to the skin, (iv) color development, (v) impact resistance (drop strength), and (vi) molding property (filling property).

In FIG. 1, the results are evaluated as “Excellent” (A), “Good” (B), “Acceptable” (C), “Poor” (D), and “Bad” (E).

In the above-described evaluation items (i) to (iv), the evaluation is conducted, depending on how many evaluation panelists of all the 5 panelists found each of the Examples better than Comparative Examples 1 and 2: 4 or 5 as “Excellent” (A); 3 as “Good” (B); 2 as “Acceptable” (C); one as “Poor” (D); and zero as “Bad” (E).

As clearly shown in FIG. 1, when a liquid paraffin or dimeticone is used as a low viscous liquid oil (C) in Example 2, use feeling and color development are evaluated highly in Example 3.

FIG. 1 shows a collection of evaluation results in comparison with the case where a prototype in which a volatile solvent (ethanol or isopropanol) is blended as a low viscous liquid oil (C) (Comparative Examples 1 and 2) is evaluated “Acceptable”.

The Examples described above can provide a wet oily solid cosmetic having excellent fixability to the skin, high clarity of application color, including a powder of a base material without whitening, and ingredients that are conspicuous on the skin and lustrous like a metal surface.

Out of the above-described 6 evaluation items, the items (v) and (vi) will be described later because they relate to a molded product produced in a later-described production method.

Method for Producing Oily Solid Cosmetic

Subsequently, the above-described method for producing an oily solid cosmetic will be described.

The above-described oily solid cosmetic is produced by stirring and mixing the above ingredients using a normal apparatus for producing cosmetics.

Specifically, a powder (A) and an oily agent (B) are first mixed so as to be uniform.

At this time, when the oily agent (B) may be an ingredient that is in the form of a solid or paste at normal temperature, it is heated and dissolved beforehand as needed for mixing. Then, a low viscous liquid oil (C) is added to a mixture of the powder (A) and the oily agent (B) for dispersion to prepare a muddy cosmetic.

For further details, the powder (A) and the oily agent (B) are mixed to prepare a cosmetic base material.

At this time, a mixer or a Henschel mixer, which is commonly used for producing a cosmetic product, is used.

The low viscous liquid oil (C) is added to the cosmetic base material and stirred by a mixer, a disperser, a homomixer or the like to obtain a muddy oily solid cosmetic. In this case, the viscosity of the low viscous liquid oil (C) is preferably 500 cSt or less, as later described in detail.

A method for producing an oily solid cosmetic of the above described Examples can provide a method for producing an oily solid cosmetic capable of producing a wet oily solid cosmetic having excellent fixability to the skin, high clarity of application color, including a powder of a base material without whitening, and ingredients that are conspicuous on the skin and lustrous like a metal surface using a powder (A), an oily agent (B), and a low viscous liquid oil (C), without using creamy solidified ingredients or volatile ingredients such as volatile solvents.

A production method of the Examples using a low viscous liquid oil (C) can advantageously require no drying step to save one production step.

Method for producing molded product using oily solid cosmetic

Subsequently, the above-described method for producing a molded product using the above-described oily solid cosmetic will be described.

An oily solid cosmetic prepared is pressed and filled from holes having a small diameter provided on the bottom of a container or the like, and solidified by providing a low viscous liquid oil (C) by vacuum upon pressing (back injection; filled from the rear surface of the container).

Specifically, after the opening of a resin dish or a metal dish is covered with an upper mold (metal mold), a muddy cosmetic is filled from holes provided on the container and pressed by providing the low viscous liquid oil by vacuum.

Further pressing is possible when the cosmetic cannot sufficiently be solidified in this step.

At this time, the cosmetic surface can be patterned by pressing with an upper mold engraved by inversion of a cosmetic surface pattern and a shape.

The cosmetic is filled and pressed by heating and melting a wax ingredient when the wax ingredient that is solid at normal temperature is blended.

The viscosity of the low viscous liquid oil (C) is preferably 200 cSt or less in view of the cosmetic fluidity.

It was confirmed that considering the lower limit of the viscosity of the low viscous liquid oil (C), an oily agent having a low viscosity of 1 cSt can be blended. The viscosity can be selected according to an intended use feeling. The lower the viscosity is, the lighter the use feeling is (slipperiness and less oil feeling), and the higher the viscosity, the heavier the use feeling and the creamier the feeling.

Accordingly, a molded product is produced using the oily solid cosmetic by means of the back injection system (filled from the rear surface of the container).

Subsequently, another method for producing a molded product using the above-described oily solid cosmetic will be described.

An oily solid cosmetic prepared is filled from an upper portion of the container, and solidified by providing the low viscous liquid oil (C) by vacuum upon pressing (front injection; filled from an upper surface of the container).

Specifically, after a predetermined amount of cosmetic is filled into a resin dish or a metal dish, the opening of the resin dish is covered with an upper mold (metal mold), and the cosmetic is pressed by providing the low viscous liquid oil (C) by vacuum (vacuum compaction). The pressing may be performed twice or more times, depending on cosmetic pressing properties.

In this case, the cosmetic is filled by heating and melting a wax ingredient when the wax ingredient that is solid at normal temperature is blended.

The viscosity of the low viscous liquid oil (C) is preferably 500 cSt or less in view of the cosmetic fluidity.

It was confirmed that considering the lower limit of the viscosity of the low viscous liquid oil (C), an oily agent having a low viscosity of 1 cSt can be blended. The viscosity can be selected according to an intended use feeling. The lower the viscosity is, the lighter the use feeling is (slipperiness and less oil feeling), and the higher the viscosity, the heavier the use feeling and the creamier the feeling.

Accordingly, a molded product is produced using the oily solid cosmetic by means of the front injection system (filled from the upper surface of the container).

When the cosmetic is filled into a resin dish, cosmetics of 2 or more colors can be filled simultaneously, and can also be filled in a resin dish having a specific shape such as a curved shape.

Subsequently, regarding a molded product, evaluation results (A) to (E) of the evaluation item of the (v) impact resistance (drop strength) will be described with reference to FIG. 1.

In this case, the evaluation is conducted according to the number of 50 cm-drops for a molded product to be cracked more or less than Comparative Examples 1 and 2: 4 or higher more as Evaluation (A); 2 or 3 more as Evaluation (B); equal (±1) as Evaluation (C); 2 or 3 less as Evaluation (D); and 4 or higher less as Evaluation (E).

Subsequently, regarding a molded product, evaluation results (A) to (E) of the evaluation item of the (vi) molding property (filling property) will be described with reference to FIG. 1.

In this case, the evaluation is conducted according to the hardness degree measurement after filling a molded product more or less than Comparative Examples 1 and 2: 20% or higher more as Evaluation (A); 10 to 19% more as Evaluation (B); equal (±10%) as Evaluation (C); 10% to 19% less as Evaluation (D); and 20% or higher less as Evaluation (E).

The method for producing a molded product using the above-described oil solid cosmetic can obtain a molded product having favorable impact resistance and molding property and suitable as eye shadow, eyebrow, cheek makeup, foundation, lipstick cosmetics and so forth, using the oily solid cosmetic having the above-mentioned advantageous effects of the invention.

### Industrial Applicability

An oily solid cosmetic and a molded product of the present invention can suitably be used as eye shadow, eyebrow, cheek makeup, foundation, lipstick cosmetics and so forth.

### DESCRIPTION OF REFERENCE NUMERALS

(A) Powder
(B) Oily agent
(C) Low viscous liquid oil

1. An oily solid cosmetic comprising:
a cosmetic base material obtained by mixing a powder and an oily agent; and
a low viscous liquid oil that is not volatile at normal temperature for rendering muddy the cosmetic base material.

2. An oily solid cosmetic comprising:
a cosmetic base material obtained by mixing a powder and an oily agent, the powder comprising a coloring agent; and
a low viscous liquid oil that is not volatile at normal temperature for rendering muddy the cosmetic base material.

3. The oily solid cosmetic according to claim 2, wherein
the powder comprising a coloring agent is blended in an amount of 20 to 65% by weight, the oily agent in an amount of 5 to 20% by weight, and the low viscous liquid oil that is not volatile at normal temperature in an amount of 30 to 60% by weight.

4. A method for producing an oily solid cosmetic, comprising the steps of:
mixing a powder and an oily agent to obtain a cosmetic base material; and
adding to the cosmetic base material a low viscous liquid oil that is not volatile at normal temperature, and stirring the resulting mixture to render muddy the cosmetic base material.

5. A method for producing an oily solid cosmetic, comprising the steps of:
mixing a powder and an oily agent to obtain a cosmetic base material, the powder comprising a coloring agent; and
adding to the cosmetic base material a low viscous liquid oil that is not volatile at normal temperature, and stirring the resulting mixture to render muddy the cosmetic base material.

6. The method for producing an oily solid cosmetic according to claim 5, wherein
the powder comprising a coloring agent is blended in an amount of 20 to 65% by weight, the oily agent in an amount of 5 to 20% by weight, and the low viscous liquid oil that is not volatile at normal temperature in an amount of 30 to 60% by weight.

7. A method for producing a molded product using an oily solid cosmetic, wherein
a cosmetic base material obtained by mixing a powder and oily agent is rendered muddy with a low viscous liquid oil that is not volatile at normal temperature, and filled into a container as the low viscous liquid oil is removed.

8. A method for producing a molded product using an oily solid cosmetic, wherein
a powder comprising a coloring agent is blended in an amount of 20 to 65% by weight, an oily agent in an amount of 5 to 20% by weight, and a low viscous liquid oil that is not volatile at normal temperature in an amount of 30 to 60% by weight, and a cosmetic base material obtained by mixing the powder comprising a coloring agent and the oily agent is rendered muddy with a low viscous liquid oil that is not volatile at normal temperature, and filled into a container as the low viscous liquid oil is removed.

9. The method for producing a molded product using an oily solid cosmetic according to claim 8, wherein
the viscosity of the low viscous liquid oil is 200 cSt or less when the oily solid cosmetic is filled from holes having a small diameter provided on the bottom of the container or the like.

10. The method for producing a molded product using an oily solid cosmetic according to claim 8, wherein
the viscosity of the low viscous liquid oil is 500 cSt or less when the oily solid cosmetic is filled from an upper portion of the container and pressed.
